(19) United States
(12) Patent Application Publication
(10) Pub. No.: US 2013/0317366 A1
(43) Pub. Date: Nov. 28, 2013

(54) INTRACAVITARY ULTRASOUND PROBE

(71) Applicant: PANASONIC CORPORATION, Osaka (JP)

(72) Inventors: Michiyo HIRAYAMA, Kanagawa (JP); Hiroshi TAKANO, Ehime (JP)

(73) Assignee: PANASONIC CORPORATION, Osaka (JP)

(21) Appl. No.: 13/957,179

(22) Filed: Aug. 1, 2013

Related U.S. Application Data
(63) Continuation of application No. PCT/JP2012/000688, filed on Feb. 1, 2012.

(30) Foreign Application Priority Data
Feb. 1, 2011 (JP) 2011-019474

(51) Int. Cl. A61B 8/00

(52) U.S. Cl. CPC A61B 8/4444
USPC 600/462

(57) ABSTRACT

An intracavitary ultrasound probe includes a housing having an inset section and a holding section and an ultrasound unit built into the insertion section. A center axis of the insertion section is offset with respect to a center of a cross section perpendicular to a longitudinal direction of the holding section. The holding section has a first outer surface portion and a second outer surface portion and a curvature of the first outer surface portion is smaller than a curvature of the second outer surface portion on the cross section perpendicular to the longitudinal direction.

This is a continuation of International Application No. PCT/JP2012/000688, with an international filing date of Feb. 1, 2012, which claims priority of Japanese Patent Application No. 2011-019474, filed on Feb. 1, 2011, the contents of which are hereby incorporated by reference.

### BACKGROUND

1. Technical Field

The present application relates to an intracavitary ultrasound probe to be used, for example, to check a growth state of a fetus and a uterine fibroid state.

2. Description of the Related Art

As disclosed in, for example, Japanese Patent Application Laid-Open Publication No. 2006-288773, a conventional intracavitary ultrasound probe includes an insertion section that includes an ultrasound unit built inside at a position close to a distal end thereof, and a holding section that has a circular cross section and is connected to a rear end of the insertion section. Japanese Patent Application Laid-Open Publication No. 2006-288773 also discloses a puncture adapter to be mounted to such an intracavitary ultrasound probe as described above.

A doctor grips the holding section, and inserts, in this state, the insertion section into a body cavity to make diagnoses. A reception echo is acquired through transmission and reception of an ultrasonic wave by the intracavitary ultrasound probe, and based on the reception echo, a tomographic image is generated and characteristic values of body tissue and the like are obtained. In this manner, the doctor checks, for example, the growth state of a fetus and the uterine fibroid state as described above.

### SUMMARY

According to a study conducted by the inventors of the present application, the conventional intracavitary ultrasound probe has a problem of poor operability. In particular, the operability of the intracavitary ultrasound probe may be poor when the intracavitary ultrasound probe is rotated about its axis to obtain diagnosis images of different regions. The present application provides an intracavitary ultrasound probe having excellent operability.

According to one exemplary embodiment of the present subject matter, there is provided an intracavitary ultrasound probe, including: a housing including: an insertion section having a center axis; and a holding section extending in a longitudinal direction of the housing, the holding section being connected to one end of the insertion section so that the center axis and the longitudinal direction are parallel to each other; and an ultrasound unit built into the insertion section at a position on another end side thereof, in which the housing includes an outer surface, in which the holding section includes: a first axis that matches with the center axis of the insertion section; and a second axis that passes through a center of a cross section taken in the vicinity of a longitudinal center of the housing, and extends in parallel to the longitudinal direction, in which, in a cross section perpendicular to the longitudinal direction of the holding section, the first axis is offset from the second axis in an offset direction, in which, in the cross section perpendicular to the longitudinal direction of the holding section, the outer surface of the holding section includes: a first outer surface portion located on a line extending from the second axis in the offset direction; and a second outer surface portion located on a line extending from the second axis in a direction opposite to the offset direction, and in which a curvature of the first outer surface portion is smaller than a curvature of the second outer surface portion.

According to the above aspect, the curvature of the first outer surface portion of the holding section is smaller than the curvature of the second outer surface portion thereof. Therefore, the intracavitary ultrasound probe can be supported stably.

Additional benefits and advantages of the disclosed embodiments will be apparent from the specification and Figures. The benefits and/or advantages may be individually provided by the various embodiments and features of the specification and drawings disclosure, and need not all be provided in order to obtain one or more of the same.

### BRIEF DESCRIPTION OF THE DRAWINGS

FIG. 1 is an exemplary side view of an intracavitary ultrasound probe according to an embodiment of the present invention.

FIG. 2 is an exemplary top view of the intracavitary ultrasound probe of FIG. 1.

FIG. 3 is an exemplary front view of the intracavitary ultrasound probe of FIG. 1.

FIG. 4 is an exemplary rear view of the intracavitary ultrasound probe of FIG. 1.

FIG. 5 is an exemplary sectional view of the intracavitary ultrasound probe of FIG. 1 that is taken along the line A-A of FIG. 1.

FIG. 6 is an exemplary sectional view of the intracavitary ultrasound probe of FIG. 1 that is taken along the line B-B of FIG. 1.

FIG. 7 is an exemplary sectional view of the intracavitary ultrasound probe of FIG. 1 that is taken along the line C-C of FIG. 1.

FIG. 8 is an exemplary sectional view of the intracavitary ultrasound probe of FIG. 1 that is taken along the line D-D of FIG. 1.

FIG. 9 is an exemplary sectional view of the intracavitary ultrasound probe of FIG. 1 that is taken along the line E-E of FIG. 1.

FIG. 10 is an exemplary sectional view of the intracavitary ultrasound probe of FIG. 1 that is taken along the line F-F of FIG. 1.

FIG. 11 is an exemplary view illustrating a state of the intracavitary ultrasound probe of FIG. 1 in use.

FIG. 12 is an exemplary view illustrating another state of the intracavitary ultrasound probe of FIG. 1 in use.

### DETAILED DESCRIPTION

The inventors of the present application have conducted a detailed study on the operability of the conventional intracavitary ultrasound probe. As a result, the inventors of the present application have discovered that the holding section of the conventional intracavitary ultrasound probe has a circular cross section, and hence the holding section is hard to rotate with fingers so that the intracavitary ultrasound probe rotates about an axis extending along a longitudinal direction of the intracavitary ultrasound probe.

Therefore, an operator such as a doctor is liable to rotate the intracavitary ultrasound probe by moving his/her wrist joint. In this case, however, the axis extending along the longitudinal direction of the intracavitary ultrasound probe is shifted, thereby causing the insertion section to turn to unintended directions.

In consideration of those problems, the inventors of the present application have conceived an intracavitary ultrasound probe having excellent operability.

According to one exemplary embodiment of the present invention, there is provided an intracavitary ultrasound probe, including: a housing including: an insertion section having a center axis; and a holding section extending in a longitudinal direction of the housing, the holding section being connected to one end of the insertion section so that the center axis and the longitudinal direction are parallel to each other; and an ultrasound unit built into the insertion section at a position on another end side thereof, in which the housing includes an outer surface, in which the holding section includes: a first axis that matches with the center axis of the insertion section; and a second axis that passes through a center of a cross section taken in the vicinity of a longitudinal center of the housing, and extends in parallel to the longitudinal direction, in which, in a cross section perpendicular to the longitudinal direction of the holding section, the first axis is offset from the second axis in an offset direction, in which, in the cross section perpendicular to the longitudinal direction of the holding section, the outer surface of the holding section includes: a first outer surface portion located on a line extending from the second axis in the offset direction; and a second outer surface portion located on a line extending from the second axis in a direction opposite to the offset direction, and in which a curvature of the first outer surface portion is smaller than a curvature of the second outer surface portion.

In the cross section perpendicular to the longitudinal direction of the holding section, the first outer surface portion may include a substantial arc, and a center of the substantial arc may be located in the vicinity of the first axis in the cross section.

The housing may include a first bulging portion in the vicinity of an end portion of the holding section on the insertion section side in the longitudinal direction, and a width of the first bulging portion in the offset direction in the cross section perpendicular to the longitudinal direction may be larger than a width of the holding section in the offset direction in the cross section taken in the vicinity of the longitudinal center.

The housing may include a second bulging portion in the vicinity of an end portion of the holding section on a side opposite to the insertion section side in the longitudinal direction, and a width of the second bulging portion in the offset direction in the cross section perpendicular to the longitudinal direction may be larger than the width of the holding section in the cross section taken in the vicinity of the longitudinal center.

The width of the first bulging portion in the offset direction in the cross section may be larger than the width of the second bulging portion in the offset direction in the cross section.

The first outer surface portion of the holding section may include a substantially straight line portion.

The outer surface has a substantially symmetrical shape across a plane including the first axis and the second axis.

Further, according to another embodiment of the present invention, there is provided an intracavitary ultrasound probe, including: a housing including: an insertion section having a center axis; and a holding section extending in a longitudinal direction of the housing, the holding section being connected to one end of the insertion section so that the center axis and the longitudinal direction are parallel to each other; and an ultrasound unit built into the insertion section at a position on another end side thereof, in which the housing includes an outer surface, in which a width of a contour of the outer surface in a cross section perpendicular to the longitudinal direction of the holding section is larger in a first direction than in a second direction orthogonal to the first direction, in which the contour in the cross section includes: a first arc centered on a first point that is located on a straight line parallel to the first direction; and a second arc centered on a second point that is located on the straight line, the second arc having a curvature larger than the first arc, and in which, in the cross section, the first point is closer to a point of intersection between the center axis of the insertion section and the cross section with respect to the second point.

The housing may include a first bulging portion in the vicinity of an end portion of the holding section on the insertion section side in the longitudinal direction, and a width of the first bulging portion in the first direction in the cross section may be larger than a width of the holding section in the first direction in the cross section taken in the vicinity of the longitudinal center.

The housing may include a second bulging portion in the vicinity of an end portion of the holding section on a side opposite to the insertion section side in the longitudinal direction, and a width of the second bulging portion in the first direction in the cross section may be larger than a width of the holding section in the first direction in the cross section taken in the vicinity of the longitudinal center.

The width of the first bulging portion in the first direction in the cross section may be larger than the width of the second bulging portion in the first direction in the cross section.

The contour in the cross section of the holding section may include a substantially straight line portion located opposite to the second point across the first point in the first direction.

The contour in the cross section may have a symmetrical shape across the straight line parallel to the first direction.

A distance between the first point and the second point may be larger than a difference between a radius of the first arc and a radius of the second arc.

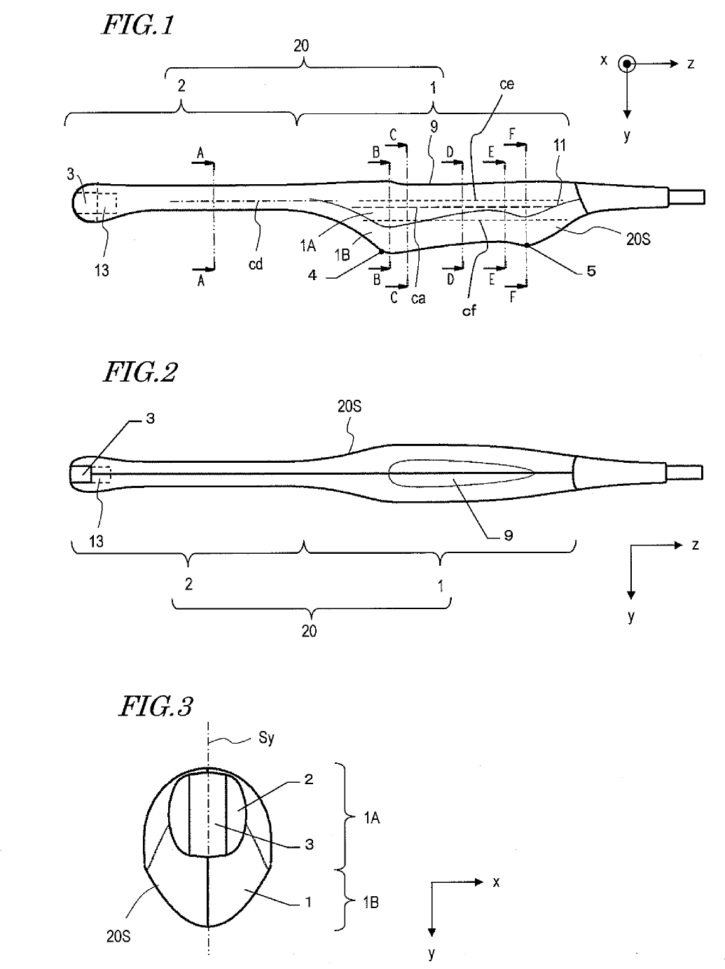

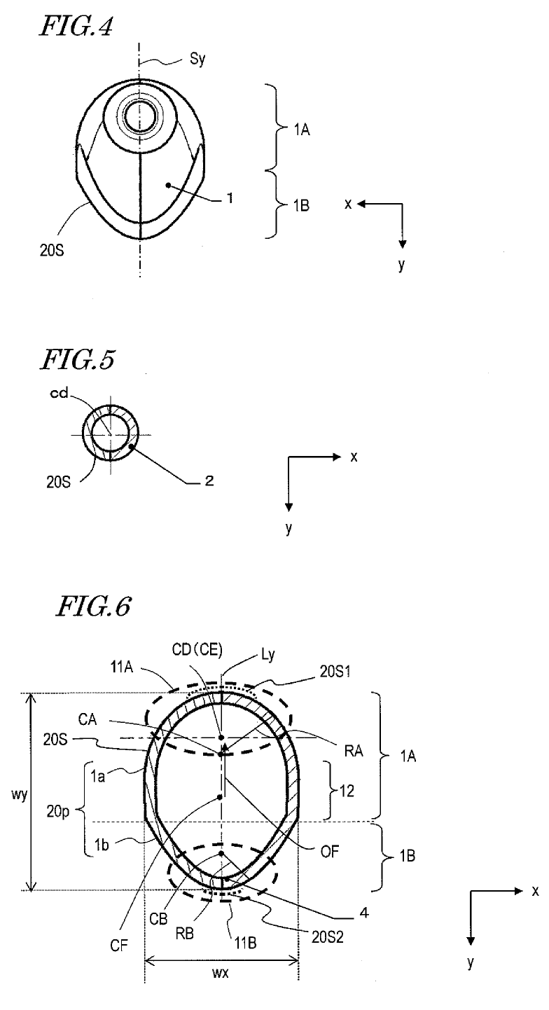

Hereinafter, an intracavitary ultrasound probe according to one embodiment of the present disclosure will be described in detail. FIG. 1 is a side view of the intracavitary ultrasound probe according to the embodiment of the present disclosure. FIG. 2 is a top view of the intracavitary ultrasound probe. FIG. 3 and FIG. 4 are a front view and a rear view of the intracavitary ultrasound probe, respectively. As illustrated in those figures, a z-axis is defined as a longitudinal direction of the intracavitary ultrasound probe, and an x-axis and a y-axis are defined in a plane perpendicular to the longitudinal direction. In the following, the intracavitary ultrasound probe is described with reference to those axes as necessary.

As illustrated in FIG. 1, the intracavitary ultrasound probe includes a housing 20 having an outer surface 20S, and an ultrasound unit 13. As illustrated in FIGS. 1 and 2, the housing 20 includes an insertion section 2 and a holding section 1. The insertion section 2 is formed into a rod shape having a center axis cd extending along the longitudinal direction (z-axis), and one end of the insertion section 2 is connected to one end of the holding section 1 so that the longitudinal direction of the holding section 1 is parallel to the center axis cd. For example, the housing 20 is made of a resin, and the insertion section 2 and the holding section 1 may be formed by molding integrally with each other.

The ultrasound unit 13 is built into the insertion section 2 at a position on another end side thereof. An acoustic lens 3 is disposed on an outer surface of the ultrasound unit 13. The ultrasound unit 13 is configured to transmit and receive an ultrasonic wave via the acoustic lens 3. As the ultrasound unit 13 and the acoustic lens 3, a conventionally known ultrasound unit and acoustic lens which are used for the ultrasound probe may be employed.

FIGS. 5 to 10 illustrate cross sections at positions indicated by the lines A-A to F-F of FIG. 1, respectively. As shown in FIGS. 5 to 10, the housing 20 has a hollow shape with a substantially uniform thickness. However, the housing 20 does not need to have a uniform thickness or a hollow shape.

As illustrated in FIG. 5, the insertion section 2 of this embodiment has a circular cross section at a center portion in the longitudinal direction, and the outer surface 20S has a circular contour in the insertion section 2. The center axis cd matches with a center of the circle defined by the contour. The insertion section 2 does not need to have a circular cross section. In this embodiment, the other end of the insertion section 2 provided with the ultrasound unit 13 has an arc-like contour in the side view of FIG. 1. Further, as illustrated in FIGS. 1, 2, and 3, at a portion provided with the ultrasound unit 13, the insertion section 2 has a larger outer shape.

As illustrated in FIGS. 1 to 4 and 6 to 10, the entire holding section 1 has a cylindrical shape or a columnar shape extending in the longitudinal direction. More specifically, the holding section 1 is formed of an upper substantially U-shaped member 1A and a lower substantially U-shaped member 1B in the cross sectional view of the holding section 1, and the entire holding section 1 has a cylindrical shape or a columnar shape extending in the longitudinal direction. Therefore, the outer surface 20S has a substantially U-shaped contour 1a and a substantially U-shaped contour 1b in an arbitrary cross section taken in a perpendicular to a longitudinal direction of the upper substantially U-shaped member 1A and the lower substantially U-shaped member 1B, respectively. Further, as illustrated in FIG. 1, the holding section 1 has a first axis ce that matches with the center axis cd of the insertion section 2, and a second axis cf that passes through a center of the cross section taken in the vicinity of the longitudinal center of the holding section 1, and extends in parallel to the longitudinal direction.

In this embodiment, the U-shape refers to such a shape that ends of two substantially straight line portions on one side are connected to each other through a line including at least an arc portion. The substantially straight line portions may be substantially parallel to each other, or ends of the two substantially straight line portions on another side may be spaced farther away from each other than the ends on one side, to thereby define a V-shape. That is, it is only necessary that the line connecting the two substantially straight line portions include the arc portion. The line portion connecting the two substantially straight line portions is referred to as “bottom portion”.

As illustrated in FIGS. 6 to 10, the upper substantially U-shaped member 1A and the lower substantially U-shaped member 1B have U-shaped openings joined to each other, to thereby define an enclosed space. Therefore, the contour 1a and the contour 1b also define an enclosed contour 20p.

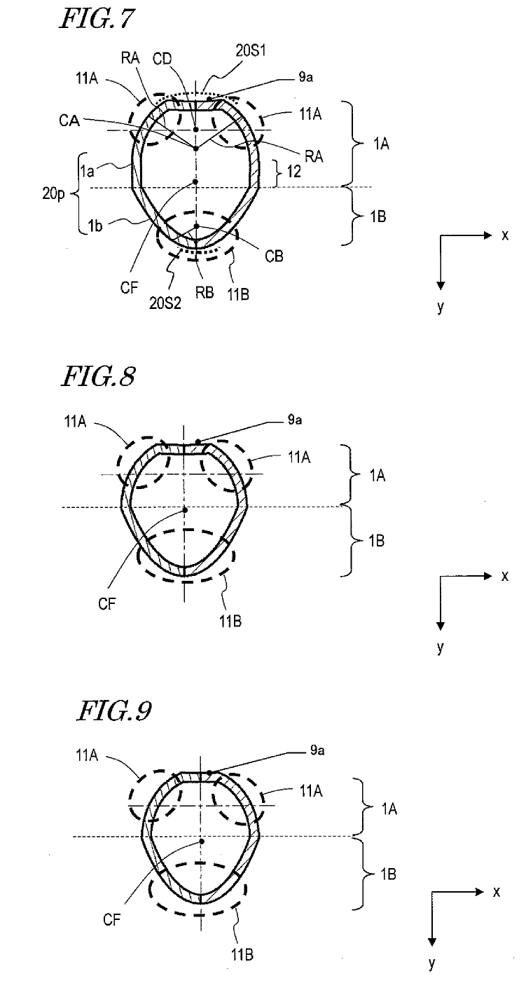

In the cross section perpendicular to the longitudinal direction of the holding section 1, the outer surface 20S, that is, the contour 20p, has a width wy in the y-axis direction, and a width wx in the x-axis direction. The width wx is smaller than the width wy. In other words, the width wy is larger than the width wx. Although the illustration is omitted in FIGS. 7 to 10 for clear understanding, in an arbitrary cross section perpendicular to the longitudinal direction of the holding section 1, the width wy may be larger than the width wx.

As illustrated in FIG. 6, in the cross section perpendicular to the longitudinal direction of the holding section 1, the contour 1a and the contour 1b have a first arc 11A and a second arc 11B, respectively. The first arc 11A is a part of a circle having a radius RA about a first point CA on a straight line Ly parallel to the y-axis, and the second arc 11B is a part of a circle having a radius RB about a second point CB on the straight line Ly. The radius RA is larger than the radius RB. Therefore, a curvature (1/RB) of the second arc 11B is larger than a curvature (1/RA) of the first arc 11A.

The first point CA is closer to a point CD with respect to the second point CB. The point CD is a point at which the center axis cd of the insertion section 2 intersects the cross section perpendicular to the longitudinal direction of the holding section 1. Further, a distance between the first point CA and the second point CB is larger than a difference between the radius of the first arc 11A and the radius of the second arc 11B.

As illustrated in FIG. 6, a point CE is defined as a point at which the cross section perpendicular to the longitudinal direction of the holding section 1 intersects the first axis ce, and a point CF is defined as a point at which the cross section perpendicular to the longitudinal direction of the holding section 1 intersects the second axis cf. Then, the point CE matches with the point CD. In this cross section, the point CE of the first axis ce is offset from the point CF of the second axis cf in a minus direction of the y-axis. This direction is referred to as “offset direction OF”. Further, in the cross section perpendicular to the longitudinal direction of the holding section 1, a part of the outer surface 20S which is located on the straight line Ly extending from the point CE in the offset direction OF is defined as a first outer surface portion 20S1, and a part of the outer surface 20S which is located on the straight line Ly extending from the point CF in a direction opposite to the offset direction is defined as a second outer surface portion 20S2. Considering the above-mentioned feature in terms of the straight line Ly, it can be said that the first arc 11A and the second arc 11B include the first outer surface portion 20S1 and the second outer surface portion 20S2 on the straight line Ly, respectively, and that a curvature of the first outer surface portion 20S1 is smaller than a curvature of the second outer surface portion 20S2. Further, the point CA as the center of the first arc 11A is closer to the point CE.

In this embodiment, in arbitrary cross sections perpendicular to the longitudinal direction of the holding section 1, the positions of the first point CA are substantially the same. Therefore, as illustrated in FIG. 1, the first point CA in an arbitrary cross section is located on an axis ca. That is, in the upper substantially U-shaped member 1A, the shape and position of the curved surface defined by the first arc 11A are substantially uniform in the longitudinal direction. On the other hand, as illustrated in FIGS. 6 to 10, in arbitrary cross sections perpendicular to the longitudinal direction of the holding section 1, the positions of the second point CB are changed. This is because, as described below, a first bulging portion 4 and a second bulging portion 5 are formed in the lower substantially U-shaped member 1B.

As illustrated in FIGS. 1 and 2, the contour 20p of this embodiment has a symmetrical shape across the straight line Ly in the cross section perpendicular to the longitudinal direction of the holding section 1. Further, the holding section 1 includes a flat surface portion 9 extending in the longitudinal direction at a part of the upper substantially U-shaped member 1A which is located at the bottom portion having the U-shape. With the flat portion 9, as illustrated in FIGS. 7 to 10, the contour 20p includes a straight line portion 9a in the cross section perpendicular to the longitudinal direction of the holding section 1. The straight line portion 9a is located at the bottom portion of the contour 1a having the U-shape, that is, located opposite to the second point CB across the first point CA in the y-axis direction. The straight line portion 9a has a substantially straight line shape. Accordingly, as illustrated in FIGS. 7 to 10, at the position ranging from the C-C cross section to the F-F cross section, the first arc 11A is divided into two parts by the straight line portion 9a. Also in this case, the curvature of the straight line portion 9a is zero or close to zero, and the first arc 11A has a smaller curvature than the second arc 11B. Thus, it can be said that the curvature of the first outer surface portion 20S1 is smaller than the curvature of the second outer surface portion 20S2 even when both the curvature of the straight line portion 9a and the curvature of the first arc 11A are compared as the curvature of the first outer surface portion 20S1 to the curvature of the second arc 11B as the curvature of the second outer surface portion 20S2.

Further, as illustrated in FIGS. 1 and 6 to 10, the housing 20 includes the first bulging portion 4 in the vicinity of the end portion of the holding section 1 on the insertion section 2 side in the longitudinal direction. As is apparent through comparison between FIG. 6 and FIG. 8, the width wy in the y-direction on the cross section on which the first bulging portion 4 is located (FIG. 6) is larger than the width wy in the y-direction on the cross section taken in the vicinity of the longitudinal center of the holding section 1 (FIG. 8). This is because, in this embodiment, the contour 1a includes an enlarged portion 12 in the first bulging portion 4 and therefore the depth of the U-shape is increased. The enlarged portion 12 may be provided to the contour 1b.

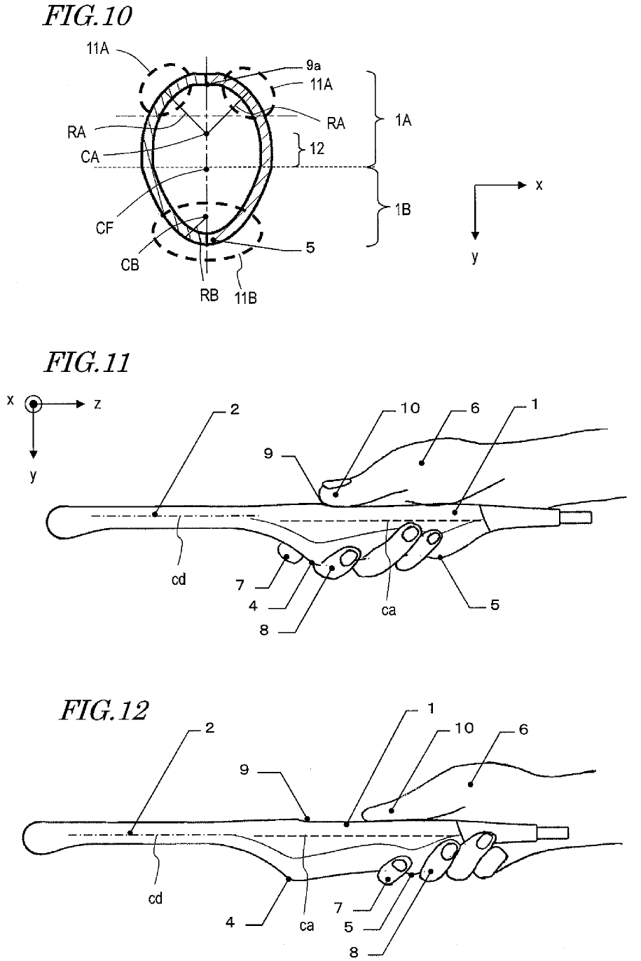

Similarly, the housing 20 includes the second bulging portion 5 in the vicinity of the end portion of the holding section 1 on a side opposite to the insertion section in the longitudinal direction. As is apparent through comparison between FIG. 8 and FIG. 10, the width wy in the y-direction on the cross section on which the second bulging portion 5 is located (FIG. 10) is larger than the width wy in the y-direction on the cross section taken in the vicinity of the longitudinal center of the holding section 1 (FIG. 8). The width wy in the y-axis direction at the first bulging portion 4 is larger than the width wy in the y-axis direction at the second bulging portion 5.

FIG. 11 illustrates a state before inserting the insertion section 2 into a body cavity by holding the intracavitary ultrasound probe of this embodiment by hand. An operator such as a doctor mainly grips the holding section of the housing 20 by hand to hold the intracavitary ultrasound probe. Specifically, for example, the operator puts his/her index finger 7 on the lower substantially U-shaped member 1B at a position on the insertion section 2 side of the first bulging portion 4, and puts his/her middle finger 8 on the lower substantially U-shaped member 1B at a position on the rear side of the first bulging portion 4. Further, the operator puts his/her ring finger and little finger on the lower substantially U-shaped member 1B at a position between the first bulging portion 4 and the second bulging portion 5 of the holding section 1.

The operator puts his/her thumb 10 and a part of his/her palm at the base of the thumb 10 on the flat surface portion 9 of the upper substantially U-shaped member 1A of the holding section 1. The palm mainly comes into contact with the half of the upper substantially U-shaped member 1A. As illustrated in FIG. 6, in the cross section in the longitudinal direction, the contour of the holding section 1 has a larger width in the y-axis direction than in the x-axis direction. Therefore, the bottom portion of the lower substantially U-shaped member 1B of the holding section 1 is easily supported at the distal ends of the fingers, in particular, in the vicinity of the distal end portions of the index finger 7 and the middle finger 8. Further, the second arc located at the bottom portion of the lower substantially U-shaped member 1B has a large curvature (small radius), and hence the bending fingers are easily fitted with the lower substantially U-shaped member 1B. On the other hand, the first arc located at the bottom portion of the upper substantially U-shaped member 1A has a small curvature (large radius), and hence the area of contact between the palm and the upper substantially U-shaped member 1A becomes larger. Thus, the intracavitary ultrasound probe can stably be supported at the upper substantially U-shaped member 1A of the holding section 1.

In this state, the operator inserts the insertion section 2 into the body cavity and then rotates the insertion section 2 inside the body cavity about the center axis cd of the insertion section 2. With this operation, an ultrasonic wave can be transmitted from the ultrasound unit 13 in arbitrary directions, and hence a reception signal can be obtained from a wide range of the body cavity.

At this time, the operator rotates the holding section 1 of the intracavitary ultrasound probe by his/her hand holding the holding section 1 of the intracavitary ultrasound probe, to thereby rotate the insertion section 2. The holding section 1 can be rotated at three points of, for example, the thumb 10, the index finger 7, and the middle finger 8. As described above, in the cross section in the longitudinal direction, the contour of the holding section 1 has a larger width in the y-axis direction than in the x-axis direction. Therefore, when a fulcrum is defined on the longitudinal axis in the vicinity of the thumb 10 put on the flat surface portion 9 located at the bottom portion of the upper substantially U-shaped member 1A, and a power point is defined on the bottom portion of the lower substantially U-shaped member 1B on which the index finger 7 and the middle finger 8 are put, a larger distance is secured from the fulcrum to the power point. Thus, based on the principle of leverage, the bottom portion of the lower substantially U-shaped member 1B is lightly moved in the x-axis direction with the index finger 7 and the middle finger 8, and accordingly the holding section 1 can be rotated about the axis extending along the longitudinal direction, with the result that satisfactory rotation operability can be obtained.

At this time, the radius of the first arc of the upper substantially U-shaped member 1A is large, and hence a part of the upper substantially U-shaped member 1A may come into contact with the palm. Further, the feeling of rotation of the upper substantially U-shaped member 1A substantially dominates the feeling of rotation of the holding section 1. Therefore, the intracavitary ultrasound probe can be rotated in a manner of sliding in contact with the palm at the part of the upper substantially U-shaped member 1A. Accordingly, the holding section 1 is easy to rotate about the axis ca on which the center of the first arc of the upper substantially U-shaped member 1A is located. The center of the first arc is located in the vicinity of the center axis cd of the insertion section 2, and hence, as described above, through the rotation of the holding section 1, the insertion section can be rotated substantially about the center axis cd thereof. Thus, the rotation axis of the holding section 1 substantially matches with the rotation axis of the insertion section 2, and hence the feeling of rotation of the holding section 1 substantially matches with the feeling of rotation of the insertion section 2, with the result that the operator can obtain an excellent feeling of operation.

FIG. 12 illustrates a case of operating the intracavitary ultrasound probe under a state in which the operator holds the rear end side of the holding section 1. Also in this case, for example, the operator may put his/her thumb 10 on the flat surface portion 9, put his/her index finger 7 on the distal end side of the second bulging portion 5, and put his/her middle finger 8 on the rear end side of the second bulging portion 5.

Thus, similarly to the holding state illustrated in FIG. 11, the intracavitary ultrasound probe can be operated at three points of the thumb 10, the index finger 7, and the middle finger 8, with the result that the operator can obtain a stable feeling of operation and satisfactory rotation operability. Further, the feeling of rotation of the holding section 1 matches with the feeling of rotation of the insertion section 2, with the result that the operator can obtain an excellent feeling of operation.

Note that, in this embodiment, the upper substantially U-shaped member 1A and the lower substantially U-shaped member 1B have the U-shaped openings joined to each other, to thereby define the enclosed space. However, the structure of the housing is not necessarily limited to the structure of the housing in which the upper and lower portions are separated from each other. It is only necessary that the outer surface have the above-mentioned shape, and as long as the enclosed space is defined, the housing may have a structure in which a C-shaped member and an inverted C-shaped member are joined to each other side by side. Alternatively, the housing may have a structure with a rectangular cylindrical shape.

The embodiment of the present invention is suitably applied to an intracavitary ultrasound probe to be used for various diagnoses. In particular, the embodiment of the present invention is suitably applied to an intracavitary ultrasound probe to be used, for example, to check a growth state of a fetus and a uterine fibroid state.

While the present invention has been described with respect to preferred embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

1-7. (canceled)

8. An intracavitary ultrasound probe, comprising:
a housing comprising:
an insertion section having a center axis; and
a holding section extending in a longitudinal direction of the housing, the holding section being connected to one end of the insertion section so that the center axis and the longitudinal direction are parallel to each other; and
an ultrasound unit built into the insertion section at a position on another end of the insertion section,
wherein the holding section has:
a first axis that matches with the center axis of the insertion section; and
a second axis that passes through a center of a first cross section perpendicular to the longitudinal direction of the holding section, and extends in parallel to the longitudinal direction,
wherein the holding section comprises:
a first holding section through which the first axis passes; and
a second holding section connected to the first holding section,
wherein, in a second cross section perpendicular to the longitudinal direction of the holding on the first axis is offset from the second axis in a first direction,
wherein, in the second cross section, an outer surface of the first holding section comprises a first curved portion having a first curvature,
wherein, in the second cross section, an outer surface of the second holding section comprises a second curved portion having a second curvature, and
wherein the first curvature is smaller than the second curvature.

9. The intracavitary ultrasound probe according to claim 8,
wherein, in the second cross section, the first curved portion comprises a substantial arc, and
wherein a center of the substantial arc is located in the vicinity of the first axis in the second cross section.

10. The intracavitary ultrasound probe according to claim 9,
wherein the housing comprises a first bulging portion disposed at a position closer to the insertion section from a longitudinal center of the holding section in the longitudinal direction, and
wherein a width of the first bulging portion in the first direction in a third cross section perpendicular to the longitudinal direction of the first bulging portion is larger than a width of the holding section in the first direction in a fourth cross section perpendicular to the longitudinal direction and taken in the longitudinal center of the holding section.

11. The intracavitary ultrasound probe according to claim 10,
wherein the housing comprises a second bulging portion disposed at a position closer to, from the longitudinal center of the holding section, an end of the housing opposite to an end of the holding section connected to the insertion section,
and
wherein a width of the second bulging portion in the first direction in a fifth cross section perpendicular to the longitudinal direction of the second bulging portion is larger than the width of the holding section in the first direction in the fourth cross section.

12. The intracavitary ultrasound probe according to claim 11,
wherein the width of the first bulging portion in the first direction in the third cross section is larger than the width of the second bulging portion in the first direction in the fifth cross section.

13. The intracavitary ultrasound probe according to claim 8,
wherein in the second cross section, the outer surface of the first holding section comprises a substantially straight line portion.

14. The intracavitary ultrasound probe according to claim 8,
wherein an outer surface of the holding section in the second cross section has a substantially symmetrical shape with respect to a plane including the first axis and the second axis.

15. The intracavitary ultrasound probe according to claim 8,
wherein, in the second cross section, the first curved portion is located on a line extending from the second axis in the first direction.

16. The intracavitary ultrasound probe according to claim 8,
wherein, in the second cross section, the second curved portion is located on a line extending from the second axis in a second direction opposite to the first direction.

17. The intracavitary ultrasound probe according to claim 8,
wherein, in the second cross section, a curvature of the outer surface of the second holding section increases from a portion connecting the first holding section and the second holding section.

18. The intracavitary ultrasound probe according to claim 8,
wherein, in the second cross section, all of curvatures of an outer surface of the holding section, when viewed from the second axis, have zero or positive values.

19. The intracavitary ultrasound probe according to claim 8,
wherein, in the second cross section, the first curved portion comprises a substantial arc, and
wherein a center of the substantial arc is located between the first axis and the second axis and closer to the first axis than to the second axis, in the second cross section.
